(19) United States
(12) Patent Application Publication
(10) Pub. No.: US 2025/0366993 A1
(43) Pub. Date: Dec. 4, 2025

(54) TRANSAPICAL VENTRICULAR MANIPULATION APPARATUS AND METHOD

(71) Applicant: Nationwide Children's Hospital, Columbus, OH (US)

(72) Inventor: Daisuke Onohara, Columbus, OH (US)

(21) Appl. No.: 19/091,136

(22) Filed: Mar. 26, 2025

Related U.S. Application Data
(60) Provisional application No. 63/569,800, filed on Mar. 26, 2024.

(51) Int. Cl. A61F 2/24; A61F 2/00

(52) U.S. Cl. CPC A61F 2/2487; A61F 2002/0081; A61F 2220/0008; A61F 2220/0075; A61F 2250/0007

(57) ABSTRACT

A transapical ventricular manipulation apparatus and method are disclosed. The apparatus for adjusting then securing portions of a heart includes a continuous tether for extending across a chamber of the heart during use, first and second anchors that are coupled by the continuous tether by passage of the tether through a substantially central opening in the first and second anchors, and first and second locking tabs, each tab having a plurality of openings for the passage of the continuous tether, the locking tabs translating along the continuous tether to engage the respective anchor to a fixed position during use.

### CROSS REFERENCES TO RELATED APPLICATIONS

The following application claims priority under 35 U.S.C. § 119(e) to U.S. Provisional Patent Application Ser. No. 63/569,800 filed Mar. 26, 2024 entitled TRANSAPICAL VENTRICULAR MANIPULATION APPARATUS AND METHOD. The above-identified application is incorporated herein by reference in its entirety for all purposes.

### STATEMENT REGARDING FEDERALLY SPONSORED RESEARCH OR DEVELOPMENT

This invention was made with government support under grant numbers HL 133667 awarded by the National Institutes of Health. The government has certain rights in this invention.

### TECHNICAL FIELD

The present disclosure generally relates to a transapical ventricular manipulation apparatus and method, and more particularly, to a device for reshaping a patient's heart, including, but not limited to improving operation, volume, and treating mitral valve regurgitation, along with a method of use and a method of construction for the same.

### BACKGROUND

A native atrioventricular heart valve can become injured and incapable of operating properly for the inability to close effectively. A typical injury is related to the structure of the heart and in particular alteration of the ventricle which is dilated.

Attempts to address and repair this type of injury or damage to the heart have been described and shown in U.S. Patent Publication Nos.: 2023/0390063 and 2023/0404760 entitled SYSTEMS, APPARATUSES, AND METHODS FOR PAPILLARY MUSCLE APPROXIMATION and DEVICE AND ASSEMBLY TO REPAIR A HEART VALVE, respectively. Both U.S. Patent Publication Nos.: 2023/0390063 and 2023/0404760 are incorporated herein in their entireties for all purposes.

### SUMMARY

In one aspect, the present disclosure includes a transapical ventricular manipulation apparatus. The apparatus for adjusting then securing portions of a heart includes a continuous tether for extending across a chamber of the heart during use, first and second anchors that are coupled by the continuous tether by passage of the tether through a substantially central opening in the first and second anchors, and first and second locking tabs, each tab having a plurality of openings for the passage of the continuous tether, the locking tabs translating along the continuous tether to engage the respective anchor to a fixed position during use.

In another aspect, the present disclosure includes an apparatus for adjusting and securing portions of a heart. The apparatus includes a continuous tether having a first end and a second end, first and second anchors that are coupled by the continuous tether by the passage of the continuous tether through an opening in each of the anchors, and first and second locking tabs each tab having a plurality of openings for the passage of the continuous tether, the locking tabs secured to the tether to allow unilateral translating along the continuous tether in only a single direction for engaging the respective anchor to a fixed position during use.

While in another aspect, the present disclosure includes a method for adjusting and securing portions of a heart comprising the steps of: inserting a first opening of a catheter through an apex of a beating heart, through a first papillary muscle, and out a first position wall of the heart; inserting a first end of a continuous tether through the catheter and out the first opening of the catheter external to the heart; removing the catheter from the apex of the heart and from the continuous tether; reinserting the first opening of the catheter through the apex of a beating heart, through a second papillary muscle, and out a second position wall of the heart; inserting a second end of the continuous tether through the catheter and out the first opening of the catheter external to the heart; and removing the catheter from the second position wall of the heart and from the continuous tether.

### BRIEF DESCRIPTION OF THE DRAWINGS

The foregoing and other features and advantages of the present disclosure will become apparent to one skilled in the art to which the present disclosure relates upon consideration of the following description of the disclosure with reference to the accompanying drawings, wherein like reference numerals refer to like parts unless described otherwise throughout the drawings and in which:

FIGS. 1-11 illustrate a method of using transapical ventricular manipulation apparatus to treat a heart of a patient in accordance with one example embodiment of the present disclosure;

FIG. 12 illustrates a heart having a mitral valve regurgitation prior to treatment of the transapical ventricular manipulation apparatus;

FIGS. 13-14 illustrate a heart correcting a mitral valve regurgitation through a treatment of the transapical ventricular manipulation apparatus and method of operation in accordance with another example embodiment of the present disclosure;

FIG. 15 illustrates components forming a transapical ventricular manipulation apparatus in accordance with another example embodiment of the present disclosure;

FIG. 16 illustrates an anchor in accordance with one example embodiment of the present disclosure;

FIG. 17 illustrates a locking tab in accordance with one example embodiment of the present disclosure;

FIG. 18 is a magnified view of one side of a heart employing transapical ventricular manipulation apparatus in accordance with one example embodiment;

FIGS. 19-24 and 26 illustrate a method of securing a locking tab with a tether of a transapical ventricular manipulation apparatus in accordance with one example embodiment; and

FIG. 25 illustrates a locking tab and anchor in situ according to another example embodiment of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures generally wherein like numbered features shown therein refer to like elements throughout unless otherwise noted. The present disclosure generally relates to a transapical ventricular manipulation apparatus and method, and more particularly, to a device for reshaping a patient's heart, including, but not limited to improving operation, volume, and treating mitral valve regurgitation, along with a method of use and a method of construction for the same.

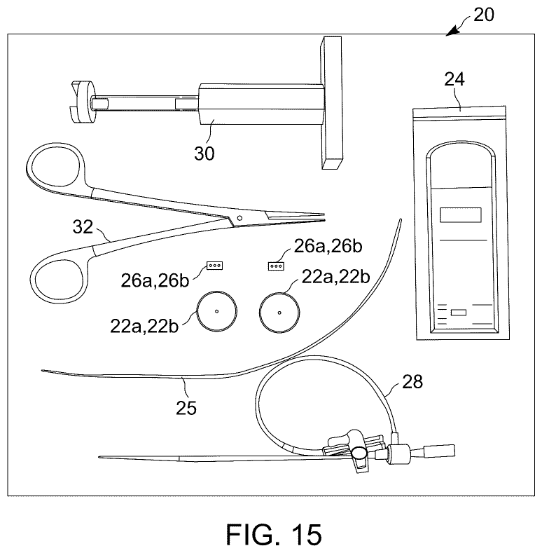

Components forming a transapical ventricular manipulation apparatus 20 are illustrated in FIG. 15 in accordance with one example embodiment of the present disclosure. In the illustrated example, the apparatus 20 comprises anchors 22a, 22b, a continuous tether (such as suture threading) 24, a cannula 25, locking tabs 26a, 26b, catheter assembly 28, puller arrangement 30, and forceps 32. It would be appreciated by those skilled in the art having reviewed this specification and figures that more of less components in FIG. 15 could be used for the apparatus 20.

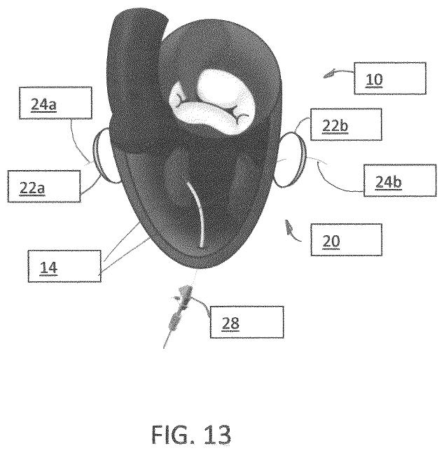

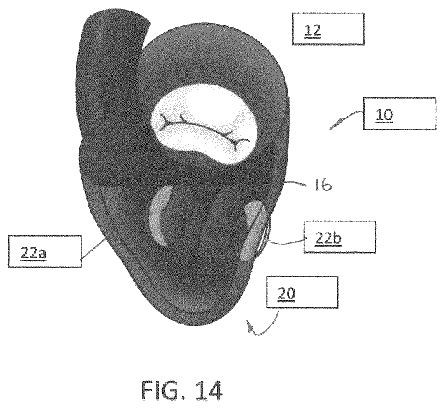

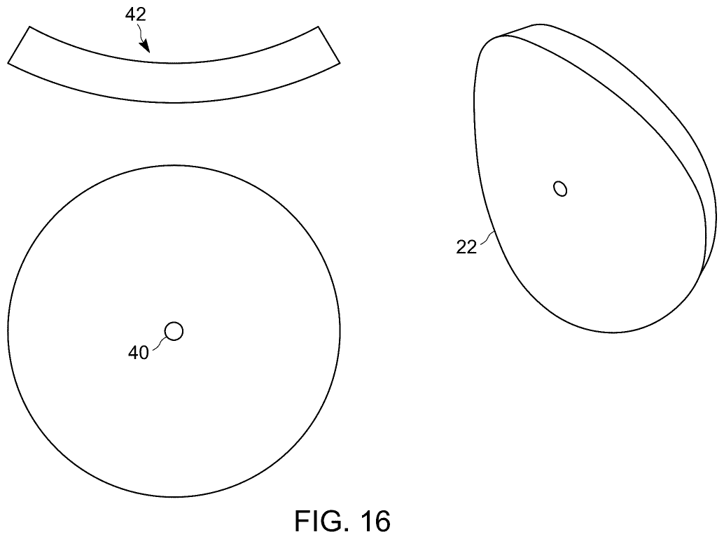

The apparatus 20, in one example embodiment, is designed to be deployed from the apex 34 of a patient's heart 10 during heartbeats. The apparatus 20 is designed such that a continuous thread or tether 24 penetrates the papillary muscle 14 and myocardium from the inner chamber to the epicardium, securing an anchor 22 having a disc-shaped geometry (see FIG. 16) on both sides of the papillary muscles 14, as illustrated in FIGS. 13 and 14. By threading a single continuous tether 24 through the apparatus 20, the anchors 22a, 22b, advantageously externally compress the left ventricle 16 towards the chamber, reshaping an enlarged left ventricle 16 back to its desirable physiological elliptical shape.

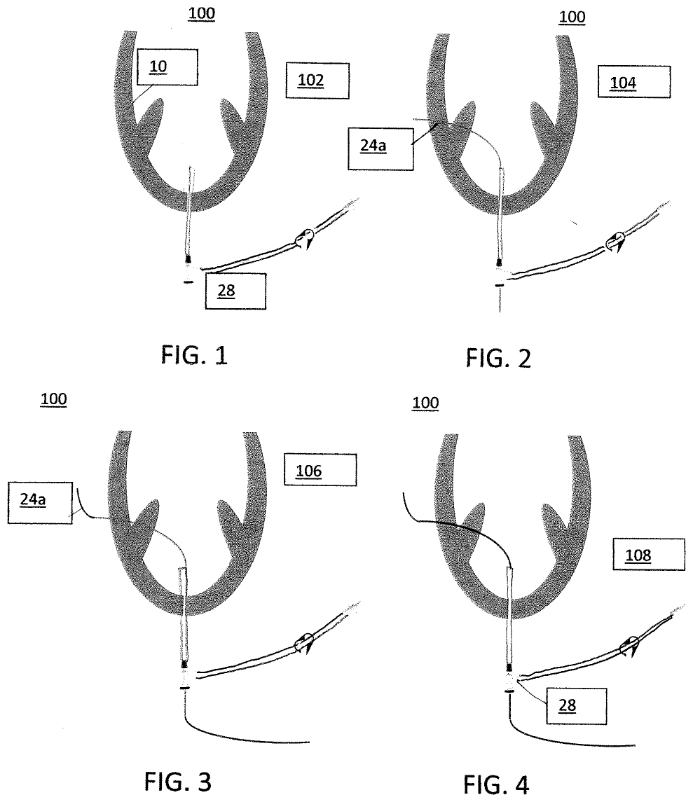

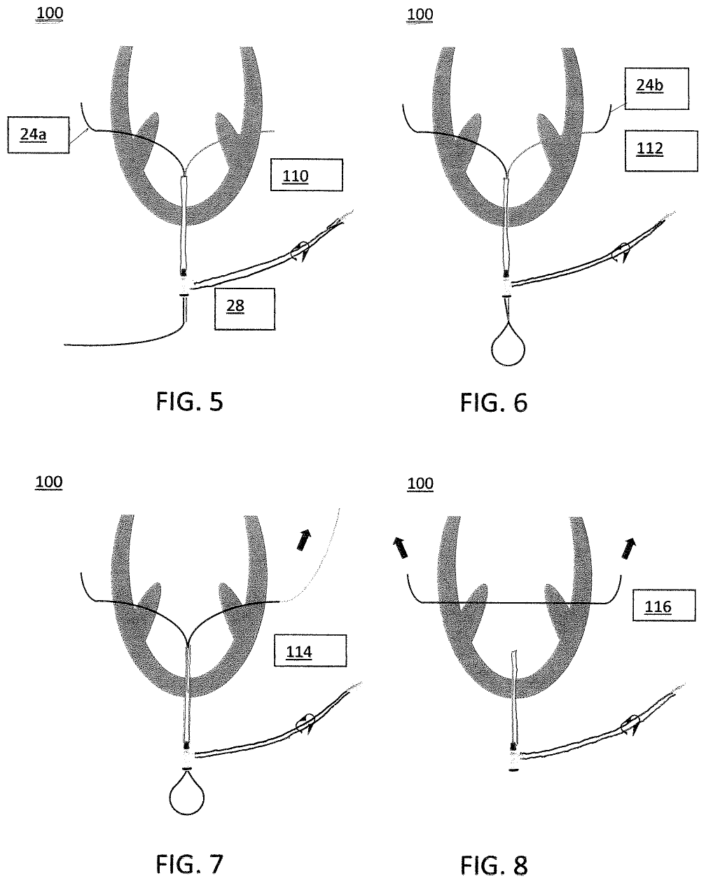

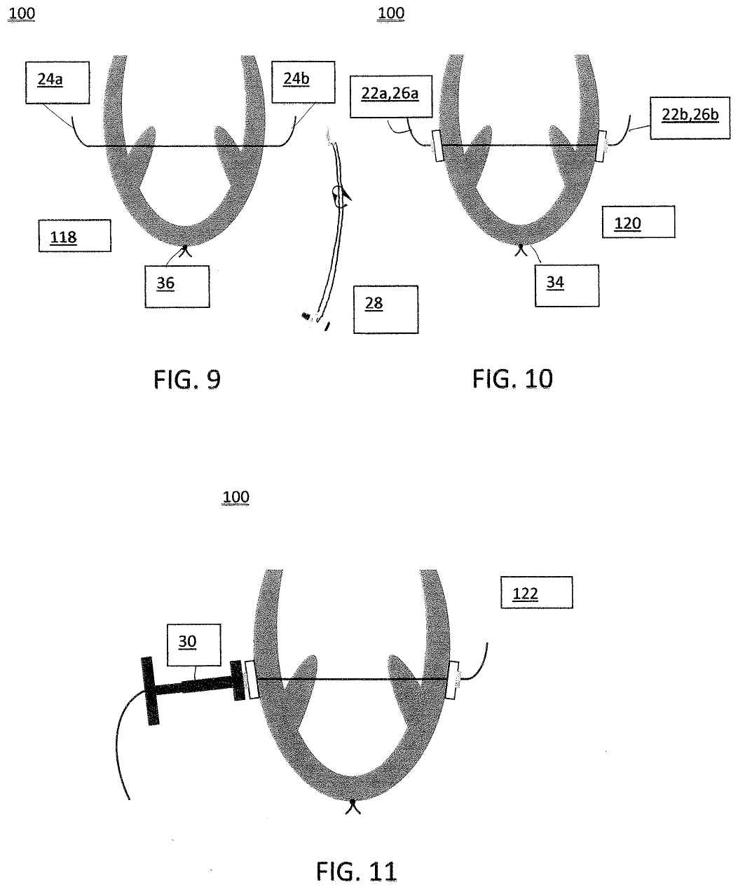

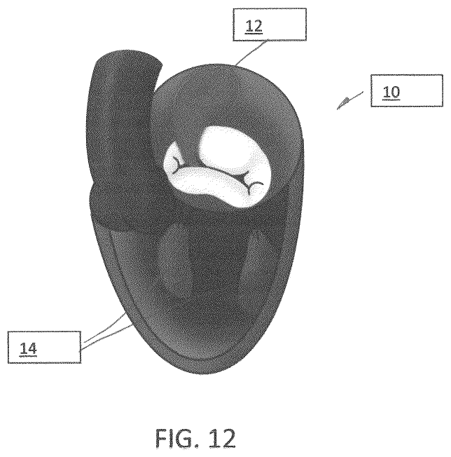

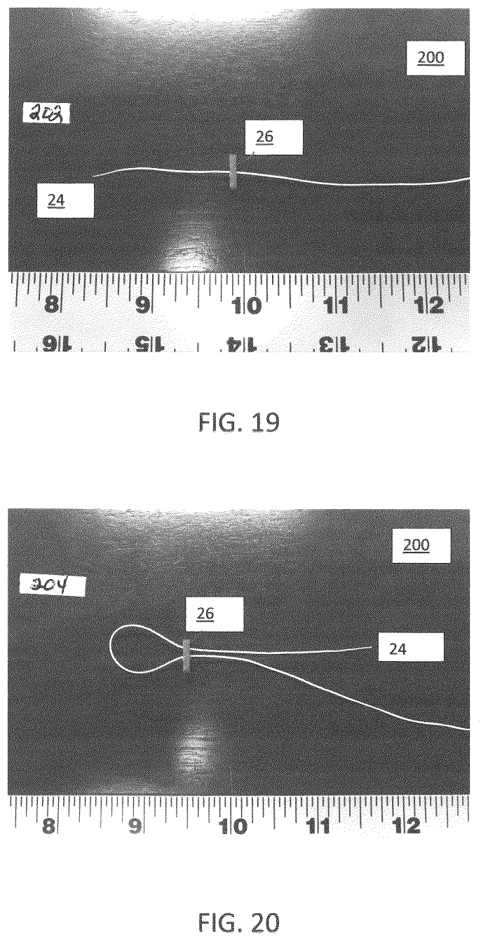

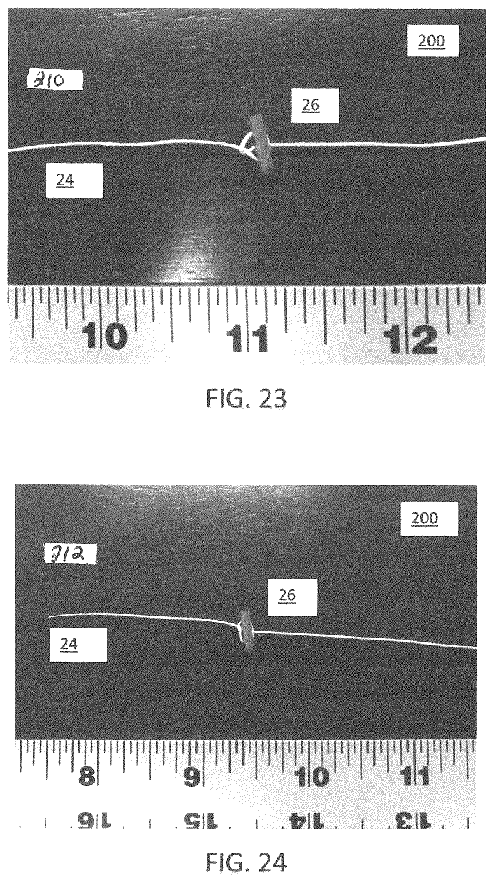

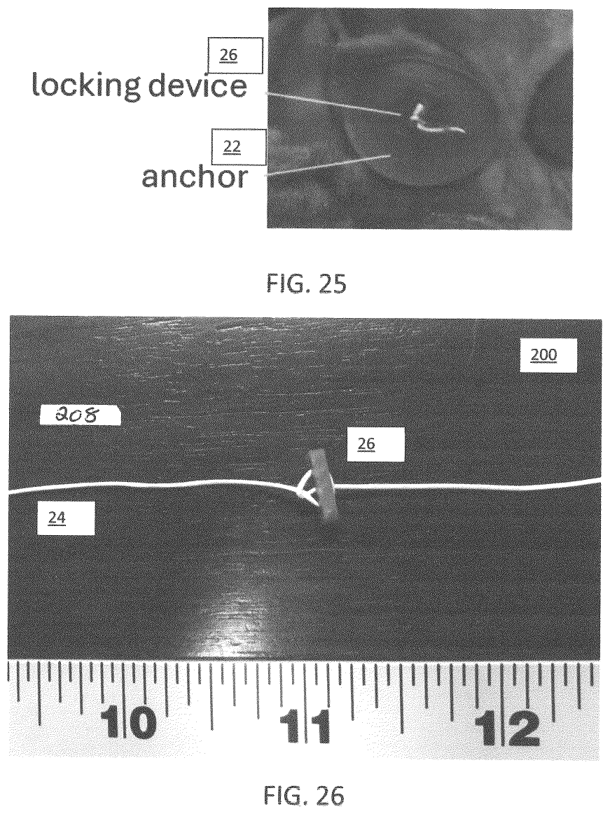

Simultaneously, the anchors 22a, 22b also approximate both papillary muscles 14 that connect with the mitral valve, not only reducing the heart's 10 volume, but also treating mitral valve regurgitation 12 (see FIG. 12 and corrected in FIGS. 13-14) through this apparatus 20. This multifaceted approach advantageously offers both an apparatus 20 and methods 100 (see FIGS. 1-11), 200 (see FIGS. 19-24 and 26) for a comprehensive solution for heart failure treatment.

In an aspect, the apparatus 20 comprises a catheter assembly 28 for introduction, anchors 22a, 22b, locking plates 26a, 26b, and thread 24. The development of the apparatus 20 and methods 100, 200 for treating heart 10 failure has substantial commercial potential and applications; for example, heart failure treatment, particularly for patients with dilated left ventricles and mitral valve regurgitation. The apparatus 20 and methods 100, 200 further provides a direct mechanical solution to reduce the volume of the left ventricle 16 and correct the shape of the heart 10, potentially improving the overall function of the heart and the patient's quality of life.

Illustrated in FIG. 16 is an anchor 22 constructed in accordance with one example embodiment. The anchor 22 is the illustrated embodiment is constructed of a biocompatible resin or polymer. In another example embodiment, the anchor 22 is created from a 3D printer.

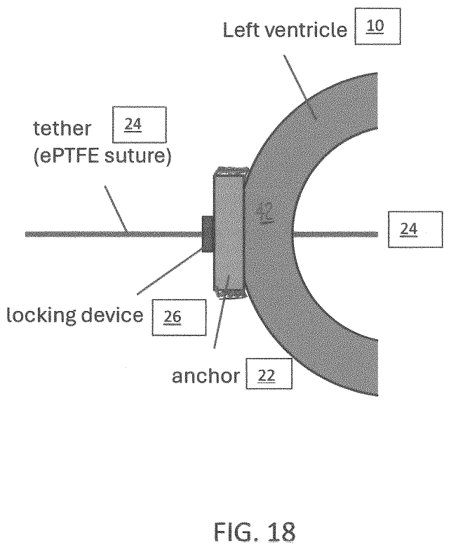

The anchor 22 includes a substantially central aperture 40 for the passage of the continuous thread or tether 24. The anchor 22 includes a arcuate surface 42 that when in situ is positioned and nests the papillary muscle 14 of the heart 10 as illustrated in FIG. 18. In one example embodiment, the anchor 22 is approximately 25 mm when pressed flat, has a thickness of approximately 3 mm and the aperture 40 is approximately 1 mm. Of course, larger and smaller dimensions of the anchor 22 are contemplated to be within the spirit and scope of the present disclosure. The anchor 22 is configured and designed in FIG. 16 to ensure secure engagement with the left ventricular myocardium of the heart 10 and allow for controlled myocardial plication as needed.

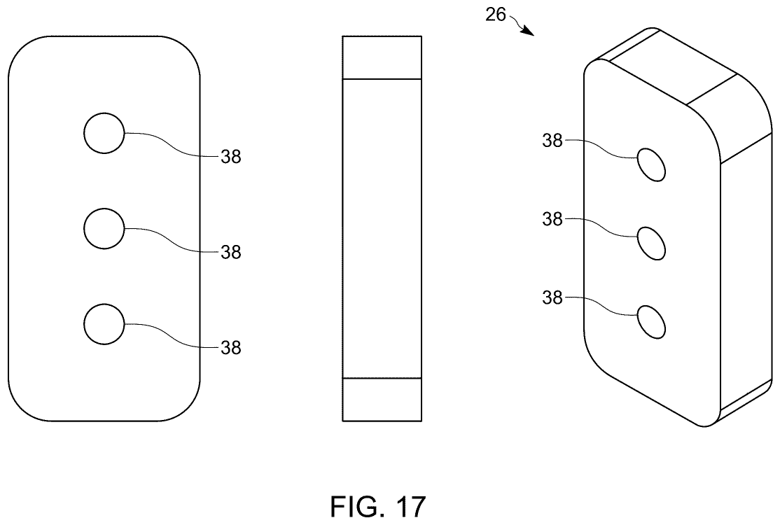

Illustrated in FIG. 17 is a locking plate or tab 26 constructed in accordance with one example embodiment. The locking plate 26 in the illustrated embodiment is constructed of a biocompatible resin or polymer. In another example embodiment, the locking plate or tab 26 is created from a 3D printer.

The locking plate 26 comprises three substantially equidistant apertures 38 for the passage of the continuous tether 24. The locking plate 26 is designed and configured to advantageously to allow the positions of two anchors 22a, 22b to be fixed and secured without the need for additional instruments.

FIGS. 1-11 illustrate a method or process 100 of using transapical ventricular manipulation apparatus 20 to treat a heart 10 of a patient in accordance with one example embodiment of the present disclosure. In particular, the process 100 illustrates the method in which a unitary continuous thread 24 is secured to portions of a patient's heart 10 while beating from the apex 34 of the heart in order to adjust portions of the heart 10 and transapical ventricular manipulation apparatus 20 to a fixed position.

The process 100 commences at 102 in which a vascular sheath of the catheter assembly 28 enters the apex 34 of the heart 10. At 104 of the process 100, a cannula or hypo-tube 25 is advanced to pass through a papillary muscle 14 through the wall of the heart 10. At step 106 of the process 100, one end the continuous thread 24a (such as a GORE-TEX suture (CV-0) is passed through the cannula 25. At step 108 the process 100, the cannula 25 of the catheter assembly 28 is removed from the apex 34 of the heart 10. At 110, the process 100 includes reinserting the cannula 25 such that it is advanced to pass through the other papillary muscle 14 through the wall of the heart 10. At 112, the process 100 includes taking an end or second end 24b opposite the first end 24a of the continuous thread 24 and looping it back into the cannula 25 until it passes out from the external wall of the heart 10. At 114, the process 100 comprises pulling the cannula 25 from the catheter assembly 28 by removing it from the second end 24b of the continuous tether 24. At 116, the process 100 includes providing tension to the first and second ends 24a and 24b, respectively in opposite directions to remove any slack in the continuous thread 24 within the heart 10. At 118, the process 100 comprises removing the catheter assembly 28 from the apex 34 of the heart 10 and forming a suture 36 to close any opening. At 120, the process 100 comprises passing anchors 22a, 22b over respective ends 24a, 24b until the arcuate surface 42 nests about the heart 10, which the anchors 22 are locked into position by respective locking plates 26a, 26b. At 122, the process employs a puller arrangement 30 in which latches to one of the ends of the continuous tether 24 to pull and position portions of the heart 10 by the location and influence of the opposite anchor 22.

FIGS. 19-24 and 26 illustrate a method or process 200 of using transapical ventricular manipulation apparatus 20 to treat a heart 10 of a patient in accordance with another example embodiment of the present disclosure. In particular, the process 100 illustrates the method in which a unitary continuous thread 24 is used to pass through a locking tab 26 in such a way to secured to portions of a patient's heart 10 while beating from the apex 34 of the heart 10 in order to adjust portions of the heart 10 and transapical ventricular manipulation apparatus 20 to a fixed position. More specifically, the process 200 is a method in which the locking plates 26a, 26b looped with an end 24a or 24b by the continuous thread 24 in a way that is designed and configured to advantageously to allow the positions of two anchors 22a, 22b to be fixed and secured without the need for additional instruments.

The process 200 commences in FIG. 19 in which one end of the continuous thread 24a or 24b after completing the process 100 of being positioned within the heart 10 passes through a first side of the locking plates 26a, 26b central aperture 38 at step 202. In this example end 24a is used while the same process occurs on the opposite side of the heart 10 with end 24b as would be appreciated by one of ordinary skill in the art after the advantage of reviewing the figures and specification of the present disclosure.

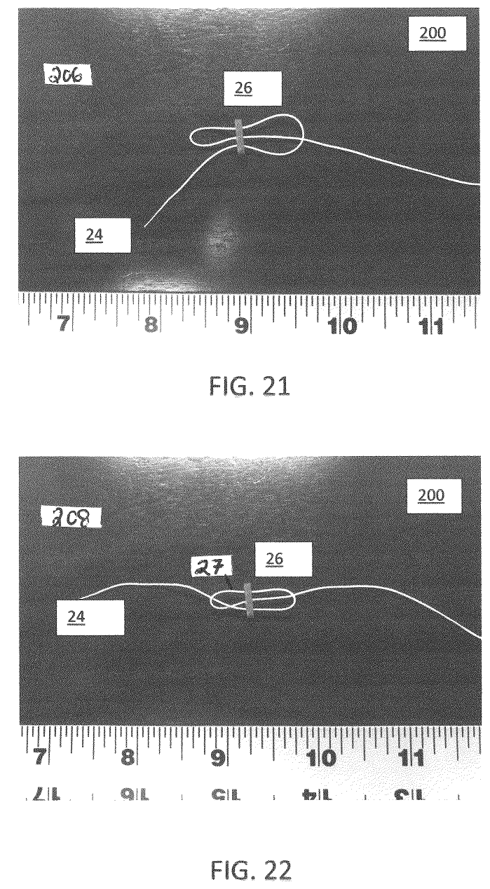

The process 200 continues in FIG. 20 at step 204 in which the end 24a then loops back through a second side opposite the first side of the locking tab 26 passing through an upper aperture 38 to form a securing loop 27. The process 200 continues in FIG. 21 at step 206 in which the end 24a loops to pass through the first side of the locking tab 26 passing through a lower aperture 38 and after exiting the second side passes through the securing loop 27 at step 208. The process 200 continues at step 210 in FIGS. 22, 23, and 26 in which the securing loop 27 is tightened to the locking tab 26. This process is repeated on the opposite side with end 22b and locking tab 26b, upon which the tabs 26 are drawn up tight against the respective anchor 22a, 22b such that the position of the heart can be manipulated to a desired position at step 212, as shown in FIG. 24.

In the illustrated example embodiments of the process 200, the heart 10 is located on the anchor 22 and locking tab 26 side opposite the securing loop of the continuous tether 24 formed in FIG. 22. By threading the continuous tether 24 through the three openings 38 and tying it in the manner described, the locking tabs 26 can be pushed in the left ventricular direction toward the anchors 22, while in the opposite direction (away from the heart), the frictional force of the securing loop 27 prevents movement, thus forming a unilateral translation arrangement. Stated another way, the securing loop 27 allows for movement of the lock toward the anchor 22 but precluded from translating in an opposite direction without fracturing or uncoupling the apparatus 20. Utilizing this method 200 of attachment, the locking tabs 26 and anchors 22 can be pushed together toward the inner chamber of the heart 10, effectively reducing the size of the dilated ventricle. This allows for the locking tabs 26 and anchors 22 to be advanced together into the heart 10 and securely fixed in place.

In one example embodiment, the apparatus 20 is a cardiac remodeling device, comprising a mechanism to reduce interpapillary muscle distance in both diastole and systole. This configuration draws the left ventricular (LV) walls in the subpapillary muscle regions toward each other. The apparatus 20 and methods 100, 200 are designed and configured to reduce functional mitral regurgitation (FMR) by addressing the tethering forces acting on the mitral valve (MV) due to a dilated LV.

The apparatus 20 and methods 100 and 200 further maintains diastolic function while achieving LV volume reduction and shape changes. The apparatus 20 and methods 100, 200 facilitate managing adverse ventricular remodeling in patients with myocardial infarction (MI) and FMR, comprising identifying patients with large LVs, reducing interpapillary muscle distance using a cardiac remodeling device, drawing in the LV walls in the subpapillary muscle regions, achieving a reduction in FMR without compromising diastolic function, and reshaping the posterior/inferior infarcted wall to enhance synchronous contraction of remaining LV segments.

In another example embodiment, the apparatus 20 and methods 100, 200 provide a system for addressing FMR and ventricular remodeling, comprising a cardiac remodeling device with components to reduce interpapillary muscle separation, a means for drawing the subpapillary muscle myocardium into the LV chamber cavity, and a mechanism to counter tethering forces on the MV caused by a dilated LV, wherein the system achieves LV volume reduction, shape changes, and improved systolic valve closure.

The apparatus 20 and methods 100, 200 in another example embodiment are adapted for treating other conditions affecting different parts of the heart 10 or for different types of heart failure, expanding its utility beyond the initial target of dilated cardiomyopathy and mitral regurgitation 12. In another example embodiment, the apparatus 20 and methods 100, 200 are employed with integrated sensor technology that incorporates sensors to monitor heart function in real-time, allowing for dynamic adjustment of the apparatus 20 or providing vital data to health care providers for ongoing patient management. For example, the apparatus 20 and/or methods 100, 200 feed into health monitoring services, offering patients and healthcare providers real-time data on heart performance and the effectiveness of the intervention. Sensors (not shown) are attached to any or all of the components forming the apparatus 20 to communicate with computers or monitors to provide feedback of a patient's heart 10 and/or condition.

In another example embodiment, the apparatus 20 and methods 100, 200 facilitate combination therapies where the apparatus 20 and methods 100, 200 are used in combination with drug therapies, providing a hybrid approach to managing heart 10 failure more effectively. In this example, the mechanical support from the apparatus 20 would enhance the efficacy of pharmaceutical treatments. Other uses of the apparatus 20 and methods 100, 200 include utilization as a research tool in clinical applications, with the apparatus 20 and methods 100, 200 serving as a valuable tool in research settings, helping scientists study the mechanics of heart failure and the effectiveness of various interventions in a controlled manner.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%.

The term “coupled” as used herein is defined as connected or in contact either temporarily or permanently, although not necessarily directly and not necessarily mechanically. A device or structure that is “configured” in a certain way is configured in at least that way but may also be configured in ways that are not listed. The term “integral” as used herein unless defined otherwise means configured in such a way that separation would require destruction to the parts or the assembly of the parts.

It should be appreciated by those of ordinary skill in the art after having the opportunity of reviewing the drawings and/or specification of the present disclosure that it may include one or more embodiments, e.g., E₁, E₂, . . . . Eₙ and that each embodiment E may have multiple parts A₁, B₁, C₁ . . . Zₙ that (without further description) could be combined with other embodiments Eₙ, embodiment parts e.g. A₁, C₁, or lack of parts originally associated with one or all embodiments Eₙ, or any combination of parts and/or embodiments thereof. It should further be appreciated that an embodiment Eₙ may include only one part e.g. A₁ or a lesser number of parts e.g. B₁, C₁ of any embodiment or combination of embodiments that was described or shown in the specification and/or drawings, respectively in ways not enumerated or illustrated.

To the extent that the materials for any of the foregoing embodiments or components thereof are not specified, it is to be appreciated that suitable materials would be known by one of ordinary skill in the art for the intended purposes after having the benefit of reviewing the subject disclosure and accompanying drawings.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. An apparatus for adjusting then securing portions of a heart comprising:
a continuous tether for extending across a chamber of the heart during use;
first and second anchors that are coupled by said continuous tether by passage of said tether through a substantially central opening in said first and second anchors; and
first and second locking tabs, each tab having a plurality of openings for the passage of said continuous tether, said locking tabs translating along said continuous tether to engage said respective anchor to a fixed position during use.

2. The apparatus of claim 1 wherein said first and second anchors each comprise a concave surface for nesting with a portion of the heart during use.

3. The apparatus of claim 1 wherein said plurality of openings in said first and second locking tabs consist of three openings in each locking tab.

4. The apparatus of claim 1 wherein said continuous tether consists of a first end and a second end.

5. The apparatus of claim 4 wherein said first end of said continuous tether forms a first securing loop with said first locking tab and said second end of said tether forms a second securing loop with said second locking tab.

6. The apparatus of claim 2 wherein said first and second anchors further comprise a convex surface opposite and spaced from said concave surface such that when assembled said first and second locking tabs contact the convex surface of each respective anchor.

7. The apparatus of claim 1 wherein said first and second locking tabs and first and second anchors are formed from one of a polymeric material and biocompatible resin.

8. The apparatus of claim 1 wherein said continuous tether and first and second locking tabs comprise a unilateral translation arrangement such that the locking tabs can only be translated in a single direction along said tether.

9. An apparatus for adjusting and securing portions of a heart comprising:
a continuous tether having a first end and a second end;
first and second anchors that are coupled by said continuous tether by the passage of said continuous tether through an opening in each of said anchors; and
first and second locking tabs each tab having a plurality of openings for the passage of said continuous tether, said locking tabs secured to said tether to allow unilateral translating along said continuous tether in only a single direction for engaging said respective anchor to a fixed position during use.

10. The apparatus of claim 9 wherein said first and second anchors each comprise a concave surface for nesting with a portion of the heart during use.

11. The apparatus of claim 9 wherein said plurality of openings in said first and second locking tabs consist of three openings in each locking tab.

12. The apparatus of claim 9 wherein said continuous tether consists of a first end extending directly to a second end.

13. The apparatus of claim 12 wherein said first end of said continuous tether forms a first securing loop with said first locking tab and said second end of said tether forms a second securing loop with said second locking tab.

14. The apparatus of claim 10 wherein said first and second anchors further comprise a convex surface opposite and spaced from said concave surface such that when assembled said first and second locking tabs contact the convex surface of each respective anchor.

15. A method for adjusting and securing portions of a heart comprising the steps of:
inserting a first opening of a catheter through an apex of a beating heart, through a first papillary muscle, and out a first position wall of the heart;
inserting a first end of a continuous tether through said catheter and out the first opening of said catheter external to the heart;
removing said catheter from the apex of the heart and from said continuous tether;
reinserting said first opening of said catheter through the apex of a beating heart, through a second papillary muscle, and out a second position wall of the heart;
inserting a second end of said continuous tether through said catheter and out the first opening of said catheter external to the heart; and
removing said catheter from the second position wall of the heart and from said continuous tether.

16. The method for adjusting and securing portions of a heart of claim 15 further comprising the step of drawing said first and second ends of said continuous tether in opposite directions to remove all slack within the beating heart.

17. The method for adjusting and securing portions of a heart of claim 16 further comprising the step of positioning a first and a second anchor on a first end and a second end, respectively of said continuous tether.

18. The method for adjusting and securing portions of a heart of claim 17 further comprising the step of positioning a first and a second locking tab on a first end and a second end, respectively of said continuous tether to secure said first and second anchors into position.

19. The method for adjusting and securing portions of a heart of claim 18 comprising the step of forming a securing loop between said first end of said continuous tether and said first locking tab and further comprising the step of forming a securing loop between said second end of said continuous tether and said second locking tab.

20. The method for adjusting and securing portions of a heart of claim 19 comprising the step of unilaterally fixing in a single direction for both said first and second locking tabs about said continuous tether with said securing loops formed between said continuous tether and said first and second locking tabs.
